(19) United States
(12) Patent Application Publication
(10) Pub. No.: US 2002/0061299 A1
(43) Pub. Date: May 23, 2002

(54) Antioxidant gene therapy for myocardial infarction

(76) Inventor: Brent Arthur French, Charlottesville, VA (US)

Correspondence Address: Brent A. French, Ph.D., 1228 Mountford Court, Charlottesville, VA, 22901, US

(21) Appl. No.: 09/988,910

(22) Filed: Nov. 19, 2001

Related U.S. Application Data
(60) Provisional application No. 60/252,066, filed on Nov. 20, 2000.

(51) Int. Cl. A61K 48/00; C12N 9/02; C12N 15/861

(52) U.S. Cl. 424/93.21; 514/44; 435/456; 435/189

(57) ABSTRACT

A method of gene therapy is described that will protect the hearts of patients against myocardial infarction. Antioxidant enzymes are known to be cardioprotective, provided that these enzymes are administered before reperfusion. However, it has not previously been practical to administer antioxidant enzymes to patients at risk for myocardial infarction because the recombinant enzymes were expensive and required continuous intravenous administration. These limitations have been overcome by using gene therapy to provide a continuous source of antioxidant enzyme. When tested in carefully controlled pre-clinical studies, antioxidant gene therapy reduced the size of myocardial infarction by >50%. This marked reduction in infarct size could mean the difference between life and death for many patients, and would improve the quality of life for every person that survived myocardial infarction. This same approach may also be used by cardiothoracic surgeons to improve the functional recovery of donor hearts after cardiac transplantation.

### CROSS-REFERENCE TO RELATED APPLICATIONS

The present application claims the benefit of the earlier filing date of the U.S. Provisional Patent Application Ser. No. 60/252,066, filed Nov. 20, 2000, which is incorporated by reference herein in its entirety.

### STATEMENT REGARDING FEDERALLY SPONSORED RESEARCH OR DEVELOPMENT

This invention was made under a contract with an agency of the United States Government: Grant Number R01 HL-58582 from the National Heart, Lung and Blood Institute of the National Institutes of Health, with additional support provided through R01 HL-43151 and R01 HL-55757.

### BACKGROUND OF THE INVENTION

Considerable evidence indicates that reactive oxygen species (ROS), such as superoxide anion (.O₂⁻) and hydrogen peroxide (H₂O₂), contribute importantly to myocardial ischemia/reperfusion injury [1]. A number of studies have shown that IV administration of antioxidant enzymes (e.g., superoxide dismutase [SOD] and catalase) can reduce infarct size; however, other studies have failed to demonstrate a protective effect [1]. These inconsistent results suggested that antioxidant enzymes had the potential to protect the heart against myocardial infarction (MI), but that experimental variables between one study and the next could greatly influence the ultimate outcome of each study. Careful examination of the pharmacology of SOD clearly showed that the enzyme had to be present in interstitial space between cells at a critical concentration in order to achieve a cardioprotective effect [8]. The cytosolic isoform of Cu/Zn-SOD was commonly used in all of these experiments. This enzyme has a very short half-life in plasma and tissue, which means that a large bolus has to be administered in bloodstream in order to obtain a short period of protection. The “size” of this narrow window of protection and the time that it occurred after an iv bolus of SOD varied from one experimental preparation to the next, leading to the variable results found in the literature. More reliable results could be obtained by administering a constant iv infusion of SOD, but this required more recombinant enzyme than most investigators could afford.

In short, the major problem with using antioxidant enzymes to protect against MI was that they needed to be given parenterally and they had very short plasma half-lives. The disclosed invention overcomes this hurdle by making use of a naturally-occurring extracellullar isoform of SOD. In contrast to the cytosolic Cu/Zn-SOD, this extracellular SOD (Ec-SOD) has been adapted by the process of evolution to function optimally in an extracellular environment. It thus has a prolonged half-life when administered to intact mammals (nearly 24 hours), and furthermore has high affinity for heparin-sulfate proteoglycans present on cellular surfaces and in the interstitial extracellular matrix. In short, the use of Ec-SOD overcomes two of the most important shortcomings of Cu/Zn-SOD (i.e., the short plasma half-life and lack of affinity for the interstitial space).

Nevertheless, the simple use of Ec-SOD does not overcome all of the problems that prevent antioxidant enzymes from being used as effective pharmaceuticals. Indeed, patents covering the use of Ec-SOD against ischemia/reperfusion (I/R) injury were granted in the early 1990's, but no formulations containing Ec-SOD have yet been tested in clinical trials. This again relates to the pharmacological properties of antioxidant protection. In order to exert a maximal protective effect, antioxidant enzymes need to be applied in an effective dose before the bulk of the ROS are generated by I/R injury. Although it is possible to classify patients at high risk for myocardial infarction (MI), it is not possible to predict when that heart attack might occur. Thus it would be necessary to administer daily injections of antioxidant enzymes in order to provide high-risk patients with maximal protection against MI. Recombinant Ec-SOD protein is produced using the same recombinant DNA technology currently being used to produce recombinant tissue plasminogen activator (t-PA). Considering the fact that a single bolus of t-PA mightcost a patient nearly $500, it is clear that daily boluses of Ec-SOD administered of a period of weeks or months will never become an economically feasible method of protecting high-risk patients against the morbidity and mortality of MI.

The disclosed invention overcomes this important economic hurdle by using gene therapy to create an endogenous source of antioxidant protection. Although numerous studies of I/R injury have utilized antioxidant enzymes prepared by recombinant techniques [3-8], no previous work has successfully used gene therapy to protect intact animals against MI. The appended experiments demonstrate that a single iv injection of gene therapy can produce cardioprotective levels of human Ec-SOD in living animals. Furthermore, the level of cardioprotection afforded by a recombinant adenovirus (Ad5) that overexpresses Ec-SOD is comparable to that provided by the gold standard of cardioprotection (ischemic preconditioning). A conscious rabbit model of MI [11-13] was used to demonstrate the utility of this therapeutic approach. This model closely mimics the human condition of heart attack, and thus overcomes limitations inherent in open-chest models that make them far less clinically relevant [14, 15].

### BRIEF SUMMARY OF THE INVENTION

This application describes a method for using antioxidant gene therapy to protect the intact mammalian heart against myocardial infarction (MI). In its preferred formulation, Ec-SOD is chosen as the antioxidant enzyme to be produced as a result of gene therapy. However, other antioxidant enzymes such as Cu/Zn-SOD, Mn-SOD, catalase, and glutathione peroxidase can also be applied using the same gene therapy techniques. Both intracellular and extracellular forms of these antioxidant enzymes (whether naturally occurring or obtained by genetic engineering) can be used to provide cardioprotection, and combinations of these enzymes may be particularly efficacious against ischemia/reperfusion (I/R) injury.

In the current specification, an example is given in which the cardioprotective antioxidant gene therapy is administered using recombinant adenovirus (Ad5) as the gene therapy vector. However, other methods of gene delivery are also envisioned provided that the alternative methods are capable of achieving adequate levels of recombinant protein in the tissue requiring antioxidant protection. Furthermore, the example cited herein applies a sufficient quantity of gene therapy vector to achieve systemic elevations of the therapeutic protein (Ec-SOD). This systemic approach may prove useful in other settings of I/R injury (involving organs such as brain, liver or kidney), or in cases where multiple organs are at risk for I/R injury (i.e., prothrombotic or embolic states that threaten blood flow to both the brain and the heart). However, the specification is not limited to systemic administration and thus encompasses targeted delivery of gene therapy to any tissue (or region of tissue) at risk of I/R injury.

### BRIEF DESCRIPTION OF THE SEVERAL VIEWS OF THE DRAWING

FIG. 1. Experimental Protocol. Four groups of rabbits were subjected to a 30-min coronary occlusion followed by 3 d of reperfusion. Three days earlier, each rabbit in groups III and IV was injected with recombinant Ad5 (2×10⁸ pfu/kg, IV). The control-treated group (group III) received an irrelevant Ad5 reporter virus (Ad5/CMV/nls-LacZ) while the gene therapy group (group IV) received an Ad5 virus carrying the cDNA encoding human Ec-SOD (Ad5/CMV/Ec-SOD). These 2 groups (groups III and IV) received IV heparin (H, 2000 U/kg) 2 h prior to, and protamine (P, 10 mg/kg) immediately before the 30-min coronary occlusion. Twenty-four hours before the 30-min coronary occlusion, rabbits in the ischemic PC group (group II) underwent six 4-min coronary occlusion/4-min reperfusion cycles. Rabbits in the untreated group (I) did not receive Ad5, heparin, protamine or ischemic PC. All rabbits were euthanized 3 d after the 30-min coronary occlusion for infarct size determination.

FIG. 2. Northern blot analysis. A: Total RNA hybridized with a riboprobe specific for human Ec-SOD mRNA. Lane 1: RNA from COS cells infected 2d previously with Ad5/CMV/Ec-SOD. Lane 2: RNA from normal COS cells. Lane 3: RNA from the liver of a normal rabbit. Lanes 4-7: RNA isolated from the livers of 4 rabbits infected 3 d earlier with Ad5/CMV/Ec-SOD. Lane 8: RNA from the heart of a normal rabbit. Lanes 9-12: RNA from the hearts of 4 rabbits infected 3 d earlier with Ad5/CMV/Ec-SOD. B: Levels of human Ec-SOD mRNA relative to endogenous levels of beta-actin mRNA. Each bar on the graph corresponds to the lane shown above in panel A.

FIG. 3. Myocardial infarct size in the four groups. Infarct size is expressed as a percentage of the region at risk of infarction. Group I: normal rabbits subjected to a 30 min coronary occlusion. Group II: rabbits protected by late ischemic PC induced 24 h prior to coronary occlusion. Group III: control rabbits injected with an irrelevant Ad5 vector 3 d prior to coronary occlusion. Group IV: rabbits protected by antioxidant gene therapy administered 3 d prior to coronary occlusion. Open circles represent individual rabbits and solid circles represent means±SEM for each group.

FIG. 4. Relationship between size of region at risk and size of MI. The graph shows individual values and linear regression lines for the four groups. In all groups, infarct size was positively and linearly related to risk region size. The linear regression equations and R values were as follows: group I, y=0.026+0.56X, R=0.80; group II, y=−0.087+0.43X, R=0.83; group III, y=−0.047+0.67X, R=0.92; group IV, y=−0.087+0.39X, R=0.79. Analysis of covariance demonstrated that the regression line for group H was significantly different from that of groups I and III, an d that the regression line for group IV was significantly different from that of groups I and III, respectively (P<0.05 for each comparison).

FIG. 5: Detailed maps of plasmids used to generate replication-deficient Ad5 vector expressing human Ec-SOD. The Ad5 gene therapy vector used to protect the hearts of intact rabbits against myocardial infarction was generated using the system described in detail by Graham and Prevec [19]. This system makes use of an Ad5 genome cloned into a derivative of the plasmid pBR322. The plasmid bearing the Ad5 genome is referred to as pJM17, and it is depicted at left in FIG. 1. In order to insert the gene of interest (i.e., the cDNA encoding human Ec-SOD) into the Ad5 genome, it was first directionally subcloned into a plasmid shuttle/expression vector (pde1Esp1B/CMV) so that the CMV immediate-early promoter drives the expression of the inserted cDNA. The resulting vector (pde1Esp1B/CMV/Ec-SOD) was then cotransfected with pJM17 into the permissive host cell line (293) to generate recombinant virus by homologous recombination. The 293 host cell line provides the E1 genes in trans, thus complementing the genetic deficiency in the replication-deficient virus. The resulting virus (Ad5/CMV/Ec-SOD) is capable of infecting cells and expressing Ec-SOD at high efficiency, but it is not capable of producing new viral particles because it lacks the necessary E1 genes.

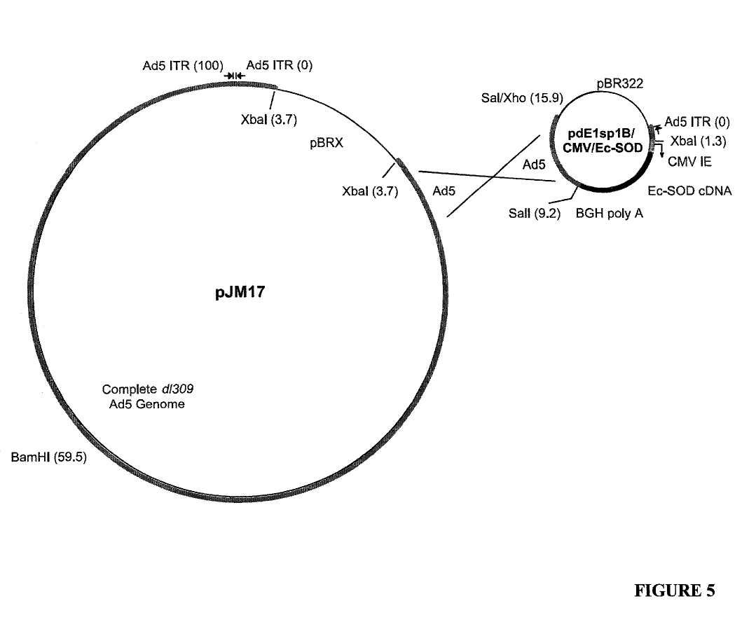

FIG. 6. Supraphysiological levels of Ec-SOD expression in vivo. Three doses of Ad5/CMV/Ec-SOD were studied in 5 rabbits (#1-#5), as indicated in the figure. Two plasma samples were drawn from each rabbit on each day—one before and one after the injection of dextran sulfate. Plasma samples were assayed for total SOD activity as described in “Methods”, and the results were expressed as total units/ml plasma. FIG. 6A illustrates the levels of SOD activity in plasma samples taken before the injection of dextran sulfate on days 0, 1, 3, 5 and 7. FIG. 6B illustrates the levels of SOD activity samples drawn 10 min after the injection of dextran sulfate (0.8 mg/kg, i.v.). Note that the y-axis of FIG. 6A is plotted on a linear scale, but that high levels of SOD activity necessitated a log scale in FIG. 6B.

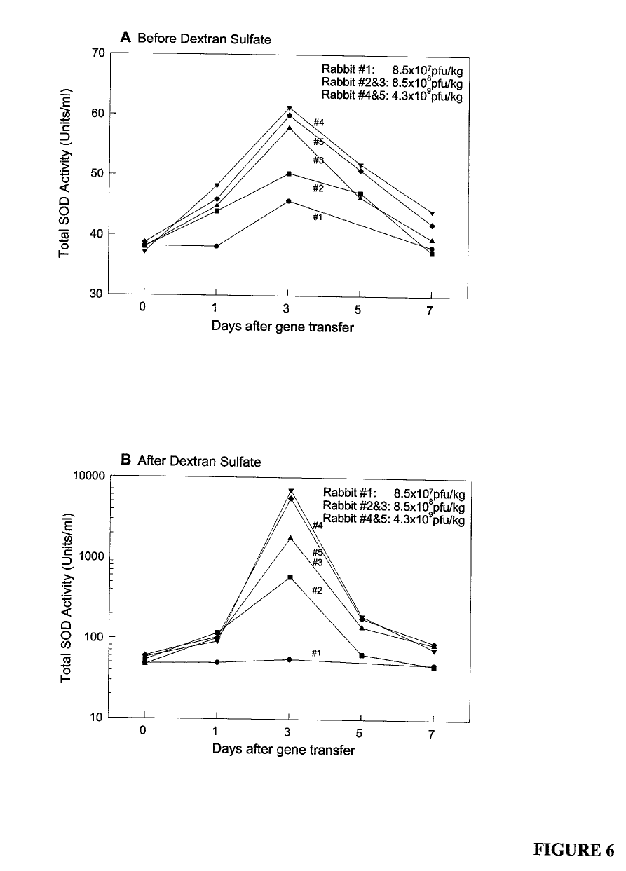

FIG. 7. Experimental protocol and measuring SOD activity in tissue samples. Panel A illustrates the experimental protocol for studying the effects of gene therapy on myocardial stunning. Two groups of rabbits were studied (groups I and II). Stage I consisted of a sequence of six 4-min coronary occlusion/4-min reperfusion cycles (6×O/R) performed on 3 consecutive days (days 1, 2 and 3). At the completion of stage I, a 12-d period was allowed to prevent late preconditioning from interfering with stage II. Group I was then injected with 2×10⁸ pfu/kg of Ad5/CMV/nls-LacZ while group II was injected with an equivalent dose of Ad5/CMV/Ec-SOD. The stage II O/R protocol was initiated 3 d later with the injection of heparin (H) followed 2 h later by the injection of protamine (P). Immediately thereafter, the same O/R protocol performed during stage I (6×O/R) was repeated again for 3consecutive days. Panel B relates the experimental protocol in Panel A to the tissue SOD levels reported in FIG. 8. Group III (BSL) serves as control. Group IV (Ec-SOD) represents group II rabbits before the injection of heparin (H) and protamine (P) on day 1 of stage II. Group V (LacZ/H+P) represents group I rabbits after the injection of H and P on day 1 of stage II. Group VI (Ec-SOD/H+P) represents group II rabbits after the injection of H and P on day 1 of stage II. Group VII (LacZ/H+P) represents group I rabbits on day 2 of stage II, and group VIII (Ec-SOD/H+P) represents group II rabbits on day 2 of stage II.

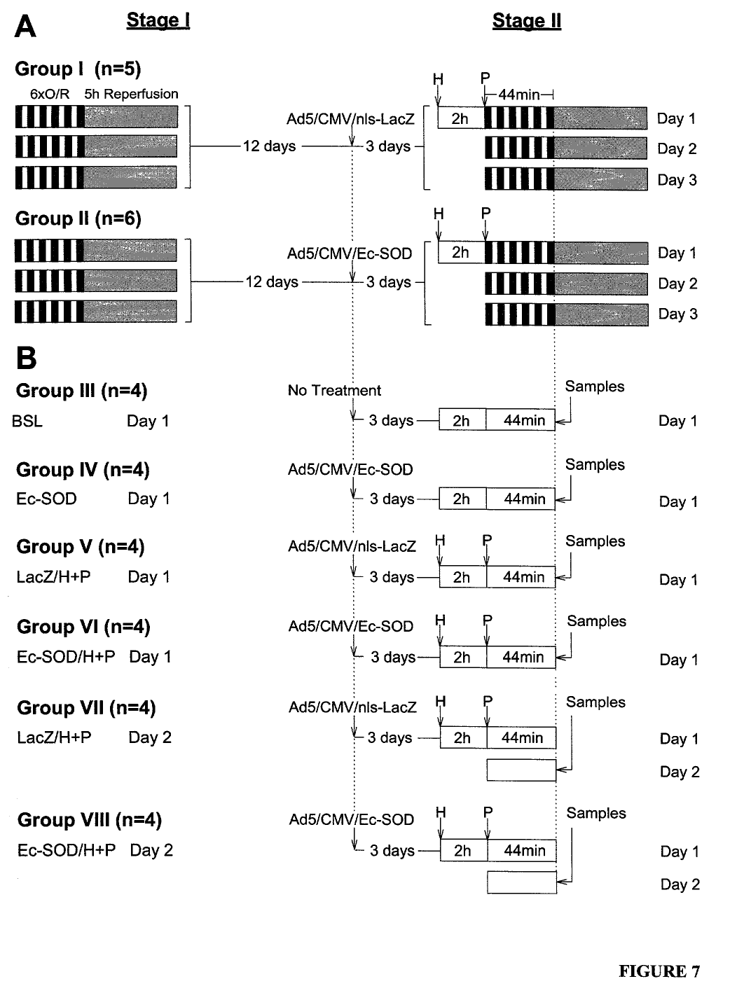

FIG. 8. Impact of Ec-SOD gene therapy on total SOD activity in liver and heart. Panel A illustrates the total SOD activities that were measured in liver samples taken from the 6 groups of rabbits defined in FIG. 7B (n=4 per group). Panel B illustrates the total SOD activities that were measured in heart samples taken from the 6 groups of rabbits defined in FIG. 7B (n=4 per group). Note that the scale used for the y-axis of Panel A is far wider than that used for Panel B because the endogenous baseline (BSL) level of total SOD activity in the rabbit liver is much higher than that found in the rabbit heart. The manipulations involving heparin (H) and protamine (P) undertaken in group II, stage II, day 1 were successful in releasing the bulk of recombinant Ec-SOD from the liver (Panel A, Group IV vs. Group VI) and redistributing a significant amount of that SOD activity to the heart (Panel B, Group IV vs. Group VI).

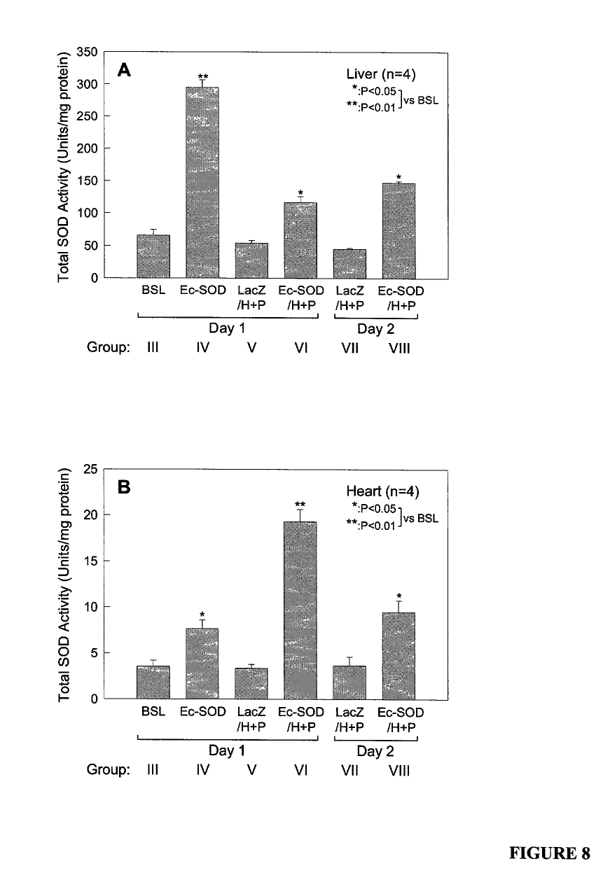

### DETAILED DESCRIPTION OF THE INVENTION

Considerable evidence indicates that reactive oxygen species (ROS), such as superoxide anion (.O₂⁻) and hydrogen peroxide (H₂O₂), contribute importantly to myocardial ischemia/reperfusion injury [1]. When examining the role of ROS, however, it is important to distinguish two forms of myocardial ischemia/reperfusion injury, namely, reversible postischemic dysfunction (myocardial stunning) and cell death (MI). Because of the fundamental differences between these two processes, pathogenetic and pathophysiological information pertaining to one of them cannot be extrapolated to the other. Indeed, while the role of ROS in myocardial stunning is generally widely accepted [2], intense controversy persists regarding whether ROS also participate in lethal ischemia/reperfusion injury (MI, [1]). A number of studies have shown that IV administration of antioxidant enzymes (e.g., superoxide dismutase [SOD] and catalase) can reduce infarct size; however, other studies have failed to demonstrate a protective effect [1]. The reasons underlying this discrepancy are unknown. We hypothesized that the inability of antioxidant enzymes to gain access to the intracellular space after IV administration may have contributed to these variable results, and postulated that the extracellular isoform of SOD (Ec-SOD), which binds to heparan sulfate proteoglycans on cellular surfaces, might provide for more consistent cardioprotection than the freely-soluble form of the enzyme.

Another problem with the use of antioxidant enzymes to protect against MI is the fact that they need to be given parenterally and have short plasma half-lives. These limitations can potentially be overcome by using gene therapy to create an endogenous source of antioxidant protection. Although numerous studies of ischemia/reperfuision injury have utilized antioxidant enzymes prepared by recombinant techniques [3-8], none of them has used gene therapy to protect intact animals against MI.

The goal of this study was to compare the protection against MI afforded by a recombinant adenovirus (Ad5) that overexpresses Ec-SOD with that afforded by the late phase of ischemic preconditioning (PC). We have previously found that Ad5-mediated gene transfer of Ec-SOD protects against myocardial stunning in conscious rabbits [9]. However, the protective effects we observed against myocardial stunning could not necessarily be extrapolated to MI, since many interventions that alleviate myocardial stunning fail to reduce infarct size [1, 2]. In both the previous and current studies, the liver was targeted for gene transfer to exploit the efficiency with which Ad5 transfects hepatocytes after IV injection, and to preclude the possibility of an inflammatory response against Ad5 [10] in the heart. A conscious rabbit model of MI [11-13] was employed to overcome limitations inherent in open-chest preparations that could interfere with the study of ischemia/reperfusion injury [14, 15].

Methods

Surgical preparation. All animal experiments were performed in accordance with institutional and federal guidelines (DHHS Publication No. [NIH] 86-23). The conscious rabbit model of MI has been described previously [11-13]. Briefly, pentobarbital anesthetized (35 mg/kg, IV) New Zealand White male rabbits (weight, 2.1±0.2 kg) were instrumented under sterile conditions with a balloon occluder placed around a major branch of the left coronary artery, a 10-MHz pulsed Doppler ultrasonic crystal [16] in the center of the region to be rendered ischemic, and bipolar ECG leads on the chest wall. Gentamicin was administered before surgery and on the 1st and 2nd postoperative days (0.7 mg/kg/d, IM). Rabbits were allowed to recover for a minimum of 14 d after surgery.

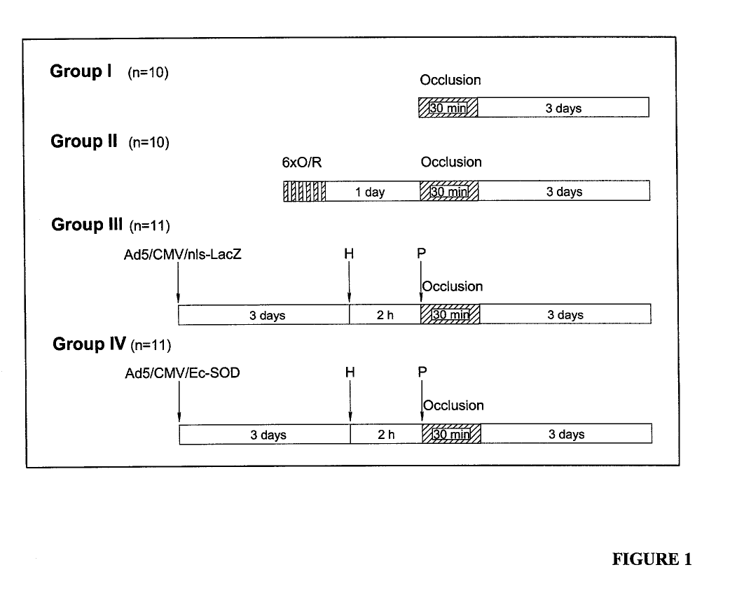

Infarction protocol. Throughout the protocol, LV systolic wall thickening (WTh), range gate depth, and the ECG were continuously recorded on a chart recorder (Gould TA6000, Valley View, Ohio). Diazepam (4 mg/kg, IP) was administered 20 min before the onset of ischemia to relieve any stress caused by coronary occlusion. No antiarrhythmic agents were administered. The infarction protocol consisted of a 30-min coronary artery occlusion followed by 3 d of reperfusion (FIG. 1). Successful occlusions were verified by observing ST-segment elevations and changes in the QRS complex on the ECGs and by paradoxical systolic wall thinning on the ultrasonic crystal recordings.

Ischemic preconditioning (PC). Ischemic PC was induced in group II with a sequence of six 4-min coronary occlusions interspersed with 4 min of reperfusion performed 24 h before the 30-min occlusion (FIG. 1). The proper execution of the occlusion/reperfusion protocol was evidenced by a 4 to 5 hour period of myocardial stunning in each animal (assessed by pulsed Doppler probe as previously described [16]).

Adenoviral vectors. The construction of the nuclear-localized LacZ reporter virus Ad5/CMV/nls-LacZ has previously been reported [17], as has the construction of the recombinant adenovirus [9] that expresses the human cDNA encoding Ec-SOD [18]. Each viral isolate was plaque-purified, verified by restriction analysis, and evaluated for its potential to overexpress enzymatic activity in 293 cells. Purified viral clones were propagated in 293 cells, isolated, concentrated and titered by plaque assay according to Graham and Prevec [19].

Expression of Ec-SOD. Ad5-mediated expression of Ec-SOD at the level of mRNA accumulation was confirmed by infecting COS cells at an MOI of 10 and harvesting the cells 2 days later for Northern analysis. Five rabbits were employed to assess the expression of Ec-SOD mRNA in vivo. One of these rabbits was an untreated rabbit which exemplified group I while the remaining four rabbits were treated with 2×10⁸ pfu/kg of Ad5/CMV/Ec-SOD 3 d prior to euthanasia to simulate the gene therapy protocol (group IV). Total RNA was extracted from tissue samples using standard procedures (RNAeasy, Qiagen Inc., Valencia, Calif.), separated by electrophoresis, and blotted onto a nylon membrane (BrightStar-Plus, Ambion Inc., Austin, Tex.). A ³²P-labeled riboprobe specific for human Ec-SOD (nt 1020 through 1389, [18]) was prepared for Northern analysis using commercial reagents (Maxiscript, Ambion Inc., Austin, Tex.). Northern blots were imaged with a Storm 840 phosphorimager (Molecular Dynamics, Sunnyvale, Calif.) and quantitated using integrated software (ImageQuaNT, version 4.2).

Experimental design. Three days before the induction of MI, rabbits were randomly assigned to one of four groups. Rabbits in either the control-treated group (group III) or the gene therapy group (group IV) were injected with 2×10⁸ pfu/kg of recombinant adenovirus via ear vein (FIG. 1). This particular dose was chosen because the same amount of Ad5/CMV/Ec-SOD had previously protected conscious rabbits against myocardial stunning [9]. In both the previous and the current studies, heparin was administered 2 h before the first occlusion to release Ec-SOD from the liver. Protamine was injected prior to coronary occlusion to reverse the effects of heparin. Both groups III and IV were subjected to this same regimen (i.e., Ad5 injection 3 d prior to coronary occlusion, heparin [2000 U/kg, IV] 2 h prior to coronary occlusion, and protamine [10 mg/kg, IV] over the last 8 min prior to coronary occlusion).

Measurement of region at risk and infarct size. At the conclusion of the study, rabbits were treated with heparin (2000 U/kg, IV), anesthetized with sodium pentobarbital (50 mg/kg, IV) and euthanized with a bolus of KCl. The heart was excised and the size of the ischemic-reperfused region (region at risk) was determined by tying the coronary artery at the site of the previous occlusion and perfusing the aortic root for 2 min with a 5% solution of phthalo blue dye at a pressure of 70 mmHg using a Langendorff apparatus [11-13]. The heart was then cut into 6-7 transverse slices, which were incubated for 10 min at 37° C. in a 1% solution of triphenyltetrazolium chloride in phosphate buffer (pH=7.4). All atrial and right ventricular tissues were removed, after which the slices were weighed, fixed in a 10% solution of neutral buffered formaldehyde, and photographed. Developed 35 mm slides were projected at 10×magnification and the borders of the infarcted, ischemic/reperfused and nonischemic regions were traced onto paper. The traces were digitized on a flatbed scanner, the corresponding areas were determined by computerized planimetry (Adobe Photoshop, Version 4.0) and infarct size was calculated as a percentage of the region at risk [11-13].

Statistical analysis. Data are reported as means±SEM. For intergroup comparisons, data were analyzed by either one-way or two-way repeated-measures ANOVA (time and group), as appropriate, followed by unpaired Student's t-tests with the Bonferroni correction. The relationship between infarct size and risk region size was compared among groups with analysis of covariance (ANCOVA) using the size of the risk region as the covariate [12]. The correlation between infarct size and risk region size was assessed by linear regression using the least-squares method. All statistical analyses were performed using the SAS software system [20].

Results

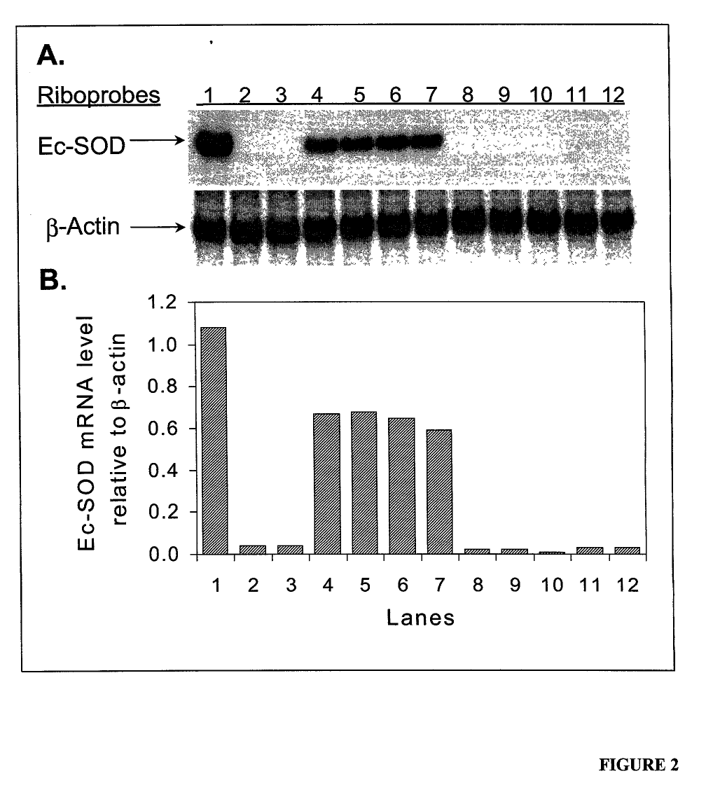

Expression of Ec-SOD. As shown in FIG. 2A, mRNA from COS cells infected with Ad5/CMV/Ec-SOD yielded a discrete band when hybridized with a riboprobe specific for human Ec-SOD mRNA (lane 1). This positive control band corresponded to abundant message in livers from rabbits treated with Ad5/CMV/Ec-SOD (lanes 4-7). No signal was detected in the liver (lane 3) or the heart (lane 8) of a normal, untreated rabbit. As anticipated, no human Ec-SOD mRNA was detected in the hearts of rabbits treated with Ad5/CMV/Ec-SOD (lanes 9-12), even though abundant message was detected in livers from these same animals (lanes 4-7). As demonstrated previously, the recombinant Ec-SOD protein is expressed in the liver, secreted from hepatocytes and then displaced by heparin for transport through the bloodstream to the heart and other organs [9]. In FIG. 2B, a quantitative analysis of the Northern blot demonstrates that IV injection of Ad5/CMV/Ec-SOD gave rise to reproducible levels of human Ec-SOD mRNA expression in the livers of experimental rabbits.

Exclusions and arrhythmias. Of the 51 rabbits instrumented, 13 were assigned to group I, 12 to group II, 15 to group III and 11 to group IV. Four rabbits died of ventricular fibrillation during coronary occlusion (2 in group I and 2 in group II). Two rabbits in group III were excluded due to technical problems with the postmortem analysis, and 3 rabbits (1 in group I and 2 in group III) were excluded because of failure of the balloon occluder. Thus a total of 10 rabbits completed the experimental protocol in group I, 10 in group II, 11 in group III, and 11 in group IV. The incidence of ventricular fibrillation during the 30-min occlusion and ventricular tachycardia following reperfusion did not differ significantly between the control and treated groups.

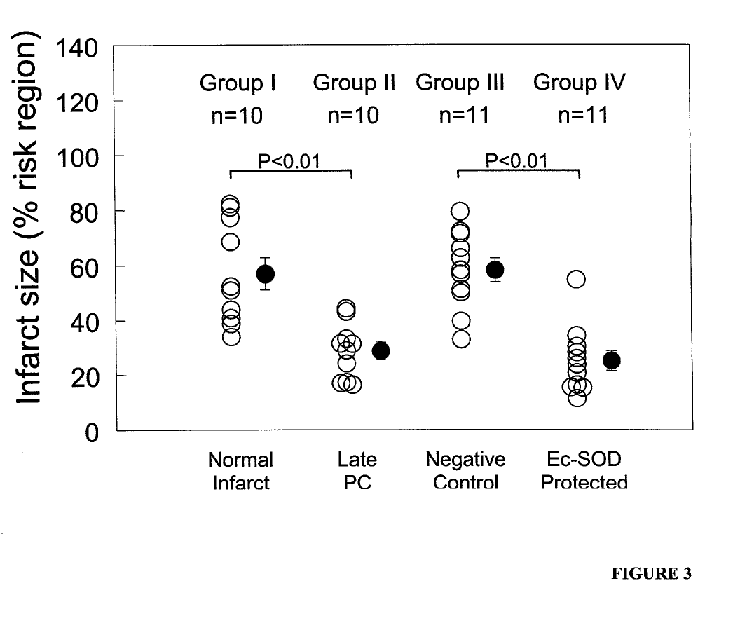

Region at risk and infarct size. On the day of the 30-min occlusion, there were no significant differences in baseline systolic thickening fraction among the four groups (39.7±2.5%, 35.7±5.1%, 35.4±2.5% and 35.7±2.6% in groups I, II, III and IV, respectively). Similarly, there were no appreciable differences among groups with respect to LV weight, region at risk, or region at risk as a percentage of LV weight. However, mean infarct size was 50% smaller in the ischemic PC group (group II) compared with the untreated group (group I) (28.6±3.2% vs. 56.9±5.9% of the region at risk, respectively; P<0.01 [FIG. 3]), indicating a late PC effect against MI. In the gene therapy group (group IV), the average infarct size was 57% smaller than in the control-treated group (group III) (25.1±4.3% vs. 58.3±5.0%, respectively; P<0.01 [FIG. 3]), indicating that the expression of Ec-SOD (as opposed to nls-LacZ) was responsible for the marked cardioprotective effect. The similarity in infarct size between the ischemic PC group (28.6±3.2%) and the gene therapy group (25.1±4.3%) indicates that the protective effect of gene therapy was comparable to that induced by the late phase of ischemic PC. The similarity in infarct size between the untreated group (56.9±5.9%) and the control-treated group (58.3±5.0%) indicates that neither the administration of an irrelevant adenoviral vector nor the injections of heparin and/or protamine had significant effects on infarct size.

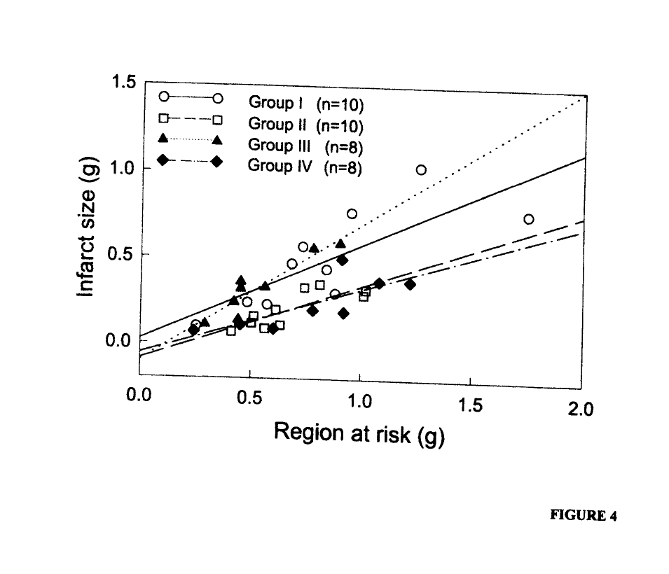

In all groups, the size of the infarction was positively and linearly related to the size of the region at risk (FIG. 4). As expected [10], the regression line was shifted to the right in the ischemic PC group as compared with the control groups (groups I and II)(P=0.05 by ANCOVA, FIG. 4). In the group pretreated with antioxidant gene therapy (group IV), the regression line was again significantly shifted to the right compared with the control groups (P<0.05 by ANCOVA, FIG. 4) and was virtually indistinguishable from that of the ischemic PC group, indicating that for any given size of the region at risk, the infarct size was reduced by antioxidant gene therapy and that the magnitude of this effect was similar to that induced by ischemic PC.

Discussion

The use of in vivo gene transfer to elevate systemic levels of therapeutic proteins has considerable potential, particularly if therapeutic levels can be achieved without adverse side-effects. The present study demonstrates that gene therapy with Ec-SOD protects conscious rabbits against MI, indicating that this approach can be effectively used to enhance endogenous antioxidant defenses and limit lethal myocellular injury during ischemia/reperfusion in vivo. While many studies have found that antioxidant enzymes can protect the heart against ischemia/reperfusion injury (reviewed in [1]), to our knowledge, this is the first demonstration that the heart can be protected against MI with the use of antioxidant gene therapy, which can provide a sustained (and potentially even permanent) supply of antioxidant proteins. The current study complements earlier work from our laboratory demonstrating that Ec-SOD gene therapy attenuates myocardial stunning in a similar conscious rabbit model [9]. Together, these studies suggest that gene therapy with Ec-SOD offers considerable potential for protecting the heart against both reversible (myocardial stunning) and irreversible (MI) ischemia/reperfusion injury. Because Ec-SOD is released into the systemic circulation and because this release can be precisely manipulated with heparin and protamine, these studies suggest a general methodological approach for controlling gene therapy at the post-translational level and for simultaneously protecting multiple tissues from ROS-mediated injury.

Methodological considerations. The rabbit model employed in this study is well characterized with respect to the infarct-sparing effects of ischemic PC [11-13], thereby providing a useful reference to compare with the effects of Ec-SOD gene therapy. The conscious preparation avoids a number of factors that could interfere with the assessment of postischemic myocardial dysfunction, such as anesthesia, surgical trauma, fluctuations in body temperature, abnormal hemodynamic conditions, elevated catecholamine levels, cytokine release, etc. [14, 15]. Most importantly, the use of a conscious model avoids the exaggerated oxyradical formation observed in open-chest animals [14], which could possibly overwhelm the antioxidant therapy under investigation.

The choice of the antioxidant enzyme was also a critical methodological consideration in the current study. Many of the animal studies that have examined the role of antioxidant enzymes in protecting the myocardium against ischemia/reperfusion injury utilized continuous IV infusion of recombinant Cu/Zn-SOD or Mn-SOD protein, and thus examined the function of intracellular enzymes delivered to the extracellular space. However, careful examination of the distribution kinetics of Cu/Zn-SOD indicates that the interstitial levels of this enzyme (rather than the plasma levels) are primarily responsible for protection against myocardial ischemia/reperfusion injury [8]. This being the case, it was reasonable to consider an isoform of SOD that has natural affinity for the interstitial space. The selection of Ec-SOD for these studies was also influenced by the fact that it is the only isoform of SOD that is secreted from cells, and is thus uniquely suited for hepatic production and systemic distribution.

The possibility of an inflammatory response against the first-generation Ad5 vector was a major factor in the decision to target gene transfer to another organ besides the heart. Unlike the heart, the liver has a profound regenerative capacity, and remarkably high frequencies of Ad5-mediated transfection (>90%) can easily be obtained by simple IV injection without compromising hepatic function [21]. Not only is the liver an opportune target for Ad5, but this strategy served to alleviate concerns regarding the possibility of inflammation in the heart and its potential impact on ischemic PC and MI.

Previous studies of the cardioprotective effects of Ec-SOD. Numerous studies have examined whether Cu/Zn-SOD and Mn-SOD can limit tissue damage after coronary occlusion [1]. However, comparatively few investigations have examined the effect of Ec-SOD on myocardial ischemia/reperfusion injury and fewer still have been conducted in intact animals. Wahlund et al. [4] reported that Ec-SOD reduced creatine kinase release in rats subjected to 10 min of coronary occlusion and 24 h of reperfusion. Hatori et al. [5] found that retroinfusion of purified, recombinant Ec-SOD protein into the great cardiac vein decreased the size of MIs in open-chest pigs subjected to coronary occlusion, and that the cardioprotection provided by the combination of catalase with Ec-SOD was no greater than that provided by Ec-SOD alone. These results are congruent with our finding that Ec-SOD was effective in protecting against MI, but the present study differs in that we examined Ec-SOD produced as a result of gene therapy in a conscious animal model of MI. Thus, the current study may bear some clinical relevance, particularly when one considers that gene therapy has the potential to provide a sustained source of antioxidant enzyme over a period of weeks or even months.

Chen et al. [7] recently found that hearts isolated from transgenic mice overexpressing human Ec-SOD exhibited enhanced postischemic myocontractile function. This cardioprotective effect is consistent with our study; furthermore, the five-fold elevation in Ec-SOD levels in this study is comparable to that obtained in our rabbit model of gene therapy [9]. However, results obtained from isolated, globally-ischemic hearts may not be as clinically-relevant as results from intact hearts in conscious animals.

Finally, previous work from our laboratory described an Ec-SOD gene therapy regimen capable of reducing myocardial stunning by approximately 50% (9). This same study determined that Ad5/CMV/Ec-SOD (at a dose of 2×10⁸ pfu/kg, IV) increased total liver SOD activity by 4.4 fold, that subsequent heparin administration led to a 2-fold increase in total plasma SOD activity, and that total SOD activity in the heart was increased 5.4-fold as a result of these manipulations.

Current study. The current report builds upon previous work [9] by applying the same Ec-SOD gene therapy regimen in a model of MI (FIG. 1). As shown in FIG. 2, Northern blot analysis of rabbits treated 3 days previously with Ad5/CMV/Ec-SOD revealed consistent levels of human Ec-SOD mRNA expression in liver tissue. As summarized in FIG. 3, the same therapeutic strategy that protected against myocardial stunning in our previous study (9) also protected the heart against irreversible postischemic dysfunction—reducing the size of MI by approximately 50%. Remarkably, this level of protection was similar to that afforded by the late phase of ischemic PC.

Taken together with our previous study [9], the finding that Ec-SOD gene therapy affords robust protection against MI implicates ROS as important mediators not only of reversible contractile dysfunction (myocardial stunning) but also of irreversible ischemia-reperfusion injury (MI). Thus, the present observations significantly broaden the potential clinical usefulness of Ec-SOD gene therapy and also have important pathophysiological implications regarding the role of ROS in lethal ischemia-reperfusion injury.

The powerful protection afforded by Ad5/CMV/Ec-SOD in this investigation was somewhat unanticipated because the effectiveness of SOD in limiting infarct size has been repeatedly questioned [1]. Indeed, while our results are consistent with previous investigations of Ec-SOD [4-8], they are seemingly in contrast with several previous studies of Cu/Zn-SOD and Mn-SOD which failed to detect significant cardioprotection against infarction (reviewed in [1]). Because of the numerous methodological differences in the experimental design of these studies, it is impossible to identify the precise reason(s) for the discrepancy. One plausible explanation involves the properties of the antioxidant enzymes examined, namely, Ec-SOD vs. Cu/Zn-SOD or Mn-SOD. While Cu/Zn-SOD and Mn-SOD are freely diffusible in the plasma compartment and in the interstitial space within the myocardium, Ec-SOD binds to heparan sulfate proteoglycans [22] present on the endothelial glycocalyx, in the extracellular matrix, and on the sarcolemma of cardiomyocytes, thereby providing effective protection against .O₂⁻ in the interstitium and on vulnerable cellular surfaces. These strategic locations of the enzyme may enable it to inactivate .O₂⁻ before it can cause extensive tissue damage. Thus, we propose that the failure of previous studies to detect a protective action of Cu/Zn-SOD and Mn-SOD in models of MI may have been caused, at least in part, by the relative inability of these proteins to gain access to injurious .O₂⁻. Another difference from previous studies is that Ec-SOD released from the liver remained in the myocardium for a sustained period of time following coronary reperfusion [9], whereas Cu/Zn-SOD and Mn-SOD are quickly cleared from the circulation.

Conclusions. The present study demonstrates that gene therapy can be used to protect conscious rabbits from MI with the use of a single antioxidant enzyme (Ec-SOD). The efficacy of Ec-SOD in protecting the myocardium against ischemia/reperfusion injury contrasts with the lack of consistent protection observed with Cu/Zn-SOD or Mn-SOD and might be attributed to the extended half-life and/or the extracellular-binding properties of this unique antioxidant enzyme [22]. The present results not only implicate ROS in the genesis of lethal ischemia/reperfusion injury in the conscious animal, but also have significant clinical implications for the development of novel cardioprotective strategies.

References

1. Bolli R. Oxygen-derived free radicals and myocardial reperfusion injury: an overview. Cardiovasc Drugs Ther. 1991;5(Suppl 2):249-268.

2. Bolli R. Mechanism of myocardial “stunning”. Circulation. 1990;82:723-738.

3. Jeroudi M O, Triana J F, Patel B S, Bolli R. Effect of superoxide dismutase and catalase, given separately, on myocardial “stunning.” Am J Physiol. 1990;259:H889-H901.

4. Wahlund G, Marklund S L, Sjoquist P O. Extracellular-superoxide dismutase type C (EC-SOD C) reduces myocardial damage in rats subjected to coronary occlusion and 24 hours of reperfusion. Free Radic Res Commun. 1992;17:41-47.

5. Hatori N, Sjoquist P O, Marklund S L, Ryden L. Effects of recombinant human extracellular-superoxide dismutase type C on myocardial infarct size in pigs. Free Radic Biol Med. 1992;13:221-230.

6. Omar B A, Gad N M, Jordan M C, Striplin S P, Russell W J, Downey J M, McCord J M. Cardioprotection by Cu,Zn-superoxide dismutase is lost at high doses in the reoxygenated heart. Free Rad Biol & Med. 1990;9:465-471.

7. Chen E P, Bittner H B, Davis R D, Folz R J, Trigt P V. Extracellular superoxide dismutase transgene overexpression preserves postischemic myocardial function in isolated murine hearts. Circulation. 1996;94(Suppl II):II-412-II-417.

8. Omar B A, McCord J M. Interstitial equilibration of superoxide dismutase correlates with its protective effect in the isolated rabbit heart. J Mol Cell Cardiol. 1991;23:149-159.

9. Li, Q., Bolli, R., Qiu, Y., Tang, X-L., Murphree, S. S., and French, B. A. Gene therapy with extracellular superoxide dismutase attenuates myocardial stunning in conscious rabbits. Circulation. 1998;98:1438-1448.

10. Yang Y, Li Q, Ertl H C, Wilson J M. Cellular and humoral immune responses to viral antigens create barriers to lung-directed gene therapy with recombinant adenoviruses. J Virol. 1995;69:2004-2015.

11. Qiu Y, Rizvi A, Tang X-L, Manchikalapudi S, Takano H, Jadoon A K, Wu W-J, Bolli R. Nitric oxide triggers late preconditioning against MI in conscious rabbits. Am J Physiol 1997;273:H2931-2936.

12. Takano H, Manchikalapudi S, Tang X-L, Qiu Y, Rizvi A, Jadoon A K, Zhang Q, Bolli R. Nitric oxide synthase is the mediator of late preconditioning against MI in conscious rabbits. Circulation. 1998;98:441-449.

13. Takano H, Tang X-L, Qiu Y, Guo Y, French B A, Bolli R. Nitric oxide donors induce late preconditioning against myocardial stunning and infarction in conscious rabbits via an antioxidant-sensitive mechanism. Circ Res. 1998:83;73-84.

14. Li X-Y, McCay P B, Zughaib M, Jeroudi M O, Triana J F, Bolli R. Demonstration of free radical generation in the “stunned” myocardium in the conscious dog and identification of major differences between conscious and open-chest dogs. J Clin Invest. 1993;92:1025-1041.

15. Bolli R. Common methodological problems and artifacts associated with studies of myocardial stunning in vivo. Basic Res Cardiol. 1995;90:257-262.

16. Bolli R, Zhu W X, Myers M L, Hartley C J, Roberts R. Beta-adrenergic stimulation reverses postischemic myocardial dysfunction without producing subsequent functional deterioration. Am J Cardiol. 1985;56:964-968.

17. French B A, Mazur W, Ali N M, Geske R S, Finnigan J P, Rodgers G P, Roberts R, Raizner A E. Percutaneous transluminal in vivo gene transfer by recombinant adenovirus in normal porcine coronary arteries, atherosclerotic arteries, and two models of coronary restenosis. Circulation. 1994;90:2402-2413.

18. Hjalmarsson K, Marklund S L, Engstrom A, Edlund T. Isolation and sequence of complementary DNA encoding human extracellular superoxide dismutase. Proc Natl Acad Sci U S A. 1987;84:6340-6344.

19. Graham F L, Prevec L. Manipulation of adenovirus vectors. In: Murray E J, ed. Methods in Molecular Biology. Vol. 7: Gene Transfer and Expression Protocols. Clifton, N J: Humana Press, 1991:109-128.

20. SAS Institute. SAS/STAT User's Guide, Release of 6.03 Edition. Cary, N C. SAS Institute, 1988;675-712.

21. Kashyap V S, Santamarina-Fojo S, Brown D R, Parrott C L, Applebaum-Bowden D, Meyn S, Talley G, PaigenB, Maeda N, Brewer H B Jr. Apolipoprotein E deficiency in mice: gene replacement and prevention of atherosclerosis using adenovirus vectors. J Clin Invest. 1995;96:1612-1620.

22. Karlsson K, Sandstrom J, Edlund A, Edlund T, Marklund S L. Pharmacokinetics of extracellular-superoxide dismutase in the vascular system. Free Radic Biol Med. 1993;14:185-190.

What is claimed is:

1. A method of protecting tissues from ischemia/reperfusion injury, said method comprising the steps of:
introducing a gene construct that encodes a therapeutic antioxidant into a patient; and
expressing the gene construct to achieve therapeutic levels of the antioxidant.

2. The method of claim 1 wherein the antioxidant is selected from the group consisting of Ec-SOD, Cu/Zn-SOD, Mn-SOD, catalase, and glutathione peroxidase.

3. The method of claim 1 wherein the gene construct is introduced into the cells of said patient through the use of a gene therapy vector based on replication-deficient adenovirus.

4. The method of claim 1 wherein the gene construct is introduced into the cells of said patient through the use of a gene therapy vector based on recombinant adenovirus-associated virus (rAAV).

5. The method of claim 1 wherein the gene construct is introduced into the cells of said patient through the use of a gene therapy vector based on plasmid DNA.

6. The method of claim 1 wherein therapeutic levels of the antioxidant are achieved by placing the expression of the gene construct under the control of a regulatable promoter.
